(19) United States
(12) Patent Application Publication
(10) Pub. No.: US 2014/0371685 A1
(43) Pub. Date: Dec. 18, 2014

(54) Cannula

(71) Applicant: Peter Troesch, Beverly Hills, CA (US)

(72) Inventor: Peter Troesch, Beverly Hills, CA (US)

(21) Appl. No.: 13/918,641

(22) Filed: Jun. 14, 2013

(51) Int. Cl. A61B 17/34; A61M 5/32

(52) U.S. Cl. CPC A61B 17/3468; A61M 5/3286; A61B 17/3421; A61B 2017/3454
USPC 604/239

(57) ABSTRACT

A cannula for use in implanting a solid object into living tissue, the cannula retaining the solid object in its interior prior to the execution of the implantation process by means of a structural impediment. The execution of the implantation process circumvents the structural impediment and allows the implant object to move from the cannula into the living tissue.

### BACKGROUND OF THE INVENTION

This invention relates to hypodermic syringes and more particularly to hypodermic syringes for implanting solid objects beneath the skin of fish, birds, animals, and humans.

The art or science of restoring or preserving health has always included the injection or withdrawal of fluids from the bodies of living things. With the development of miniaturized mechanical and electrical devices that serve therapeutic purposes and more prosaic purposes such as tracking and identification, the implantation of solid things within a body has become a reality. The implantation processes first utilized surgical procedures but as miniaturization techniques became more and more effective, the surgical approach has given way to the use of devices modeled on the hypodermic syringe for injecting fluids.

The hypodermic syringe has long been used to aspirate or inject fluids for diagnostic or therapeutic purposes. It consists of a barrel which constitutes a fluid reservoir, a cannula for insertion into the body, the cannula being connected in a leak-proof way to the barrel, and a plunger that slides within the barrel and either pushes the fluid in the barrel through the canula and into the body or pulls fluid from the body into the barrel by means of an induced vacuum in the barrel.

The adaptation of the conventional hypodermic syringe for the implantation of solid objects in living bodies has taken a rather predictable path. The adaptation has focused on objects that are elongated and usually cylindrical that will slide within a more-or-less conventional cannula. A pusher rod that slides within the cannula and pushes on the object during the implantation process is attached to the plunger and the sliding seal between the plunger and the barrel is removed, thereby permitting air trapped in the barrel by the plunger to escape around the plunger.

A typical hypodermic syringe for the implantation of solid objects in living bodies is shown in FIG. 1. It consists of barrel 1, plunger 3, gasket 5, hub 7, and cannula 9. The pointed distal end of the cannula is provided by a bevel obtained by means of a bevel grinder. A cylindrical object 11 is shown in place in the cannula ready to be implanted into a body.

Plastic plunger 3 freely slides within barrel 1. The presence of synthetic rubber gasket 5 provides a user with the feel of a conventional fluid-injecting hypodermic syringe. The purpose of the gasket is to provide a frictional force that resists the movement of the plunger, just like the fluid-tight seal in a fluid-injecting syringe. Since there is no need for a leak-proof seal for a solid-object-implanting syringe, the gasket can be made of a porous material or air channels can be incorporated in the gasket to allow air to pass freely through the gasket, thereby avoiding air pressure build-up in the barrel that might force air through the cannula and the incision in the body during the implantation procedure.

If the gasket 5 is made of a material that provides a gas-tight seal, the air-escape channel 13 beneath the gasket allows air trapped in region 15 of the barrel 1 to escape to region 17 and the outside environment. The push-rod portion 19 of the plunger 3 freely slides within the cannula 9.

The metal hub 7 attaches to the barrel 1 by means of a standard Luerlock connector. The cannula 9 is held securely within the hub as a result of crimping the hub.

The barrel 1 is a conventional 3-cm³ plastic hypodermic syringe barrel like those used in fluid-injecting hypodermic syringes and is commercially available from a number of sources.

An enlarged sectional view of the plunger 3 taken on the plane 2-2 of FIG. 1 is shown in FIG. 2. The four fins 21 in freely-sliding contact with the inside surface of the barrel 1 serve to guide the plunger along a straight-line path. The region 17 of the barrel 1 through which the air trapped in the barrel behind the gasket 5 escapes to the outside environment is shown in the context of the plunger.

An enlarged side view of the portion of the plunger containing the gasket channel 23 but without the gasket 5 is shown in FIG. 3. The air-escape channel 13 is shown extending through the walls 25 and 27 of the gasket channel 23.

Several different techniques have been used to hold the implant object 11 securely within stainless-steel cannula 9. One technique is to place a compressible plastic cap over the distal end of the implant object. When the implant object is inserted into the distal end of the cannula 9, the cap becomes wedged between the implant object and the cannula thereby holding the implant object within the cannula by frictional forces. There is a short throw in injecting the implant object thereby providing better control of the implant process. However, the plastic cap accompanies the implant object into the animal. This technique also requires the implant object to be loaded into the cannula from the distal end which tends to be more difficult. Also, the inside diameter of the cannula has to be significantly larger than the implant object diameter to enable the entry of the plastic cap into the interior of the cannula.

Another technique is to hold the implant object mechanically (by means of a washer-type device) within the barrel near the point of connection of the cannula to the barrel. With this approach the plunger must be pushed much further to inject the implant object into the animal with the accompanying problem of achieving precise control of the implant process. Also, the passage of the implant object from barrel to cannula may make the injection process difficult at times. This technique is attractive however, in spite of these difficulties during the injection process, in that there is little risk in damaging the cannula point when the implant object is loaded into the barrel prior to the attachment of the cannula to the barrel.

Still another technique for holding an implant object in a cannula is by crimped regions that exist at four locations spaced at 90-degree intervals around the circumference of the cannula. This technique is best described with the aid of the two orthogonal views of cannula 9 shown in FIG. 4. The cannula is designed to handle cylindrical implant objects having outside diameters falling within a range defined by a minimum outside diameter (minOD) and a maximum outside diameter (maxOD). The minimum inside diameter (minlD) that a cannula is permitted to have is made just enough larger than the maxOD as to allow an implant object having a maxOD outside diameter to move back and forth without binding in a cannula having a minlD inside diameter. Slot 29 has a length approximately equal to two-thirds of the nominal length of the implant object. The width of the slot is equal to no more than one-half and no less than one-third the nominal diameter of the implant object. Crimps 31 are placed in the middle portion of the cannula.

The four crimp regions 31 of FIG. 4 (the one on the underside does not show) extend from the closed end of the slot 29 for a distance equal to about one-quarter of the nominal diameter of the implant object. The crimp regions taper inward so that the distance between opposing crimp regions is greater than the maxOD of the implant object at the slot end and is less than the minOD at the terminal ends of the crimp regions. With this dimensional arrangement, any implant object satisfying the minOD and maxOD requirements can be pushed into the cannula and be held securely by the crimp regions at some position within the crimp regions.

An alternative embodiment of the cannula omits the crimp regions 31 and utilizes the existence of slot 29 as a means for holding the implant object in the cannula. The maxID of the cannula is chosen to be slightly less than the minOD of the implant object. Then, as an implant object satisfying the minOD and maxOD requirements is pushed into the open end of the cannula, the edges of the slot spring apart and hold the implant object in the cannula until it is implanted into a body.

This technique also requires the implant object to be inserted into the cannula from the distal end thereby risking damage to the tip of the cannula and its cutting edges. Also, in the crimp-region version of the technique, the length of the cannula must be greater to provide room for the crimp regions.

None of the current cannula designs are completely satisfactory insofar as retaining the implant object within the cannula prior to executing the implantation process.

### BRIEF SUMMARY OF THE INVENTION

The invention is a cannula with a structural impediment which prevents an implant object from falling out of the cannula at the distal end as a result of changes in cannula orientation prior to the execution the implantation process.

The cannula is adapted to receive an implant object into its interior at the proximal end, the cannula being attachable to an implantation device having a push-rod which enters the cannula at its proximal end. An operator utilizes the implantation device to make an incision in a body with the cutting edge of a bevel at the distal end of the cannula. The operator completes the implantation process by pushing on the push-bar thereby causing a force to be applied to the implant object which results in the implant object moving from a position within the cannula out through the distal end and into the body.

The structural impediment which prevents the implant object from falling out of the cannula at the distal end as a result of changes in cannula orientation prior to the execution of the implantation process is automatically circumvented during the execution of the implantation process.

### BRIEF DESCRIPTION OF THE SEVERAL VIEWS OF THE DRAWINGS

FIG. 1 is a side view of a prior-art hypodermic syringe for implanting solid objects.

FIG. 2 is a sectional view of that portion of the plunger that resides in the barrel.

FIG. 3 is a side view orthogonal to the side view of FIG. 1 showing the portion of the plunger containing the gasket recess.

FIG. 4A is a top view of the prior-art cannula.

FIG. 4B is a side view of the prior-art cannula.

FIG. 5A is a sectional view of a first preferred embodiment of the invention.

FIG. 5B is a side view of a first preferred embodiment of the invention.

FIG. 6A is a sectional view of a second preferred embodiment of the invention.

FIG. 6B is a side view of a second preferred embodiment of the invention.

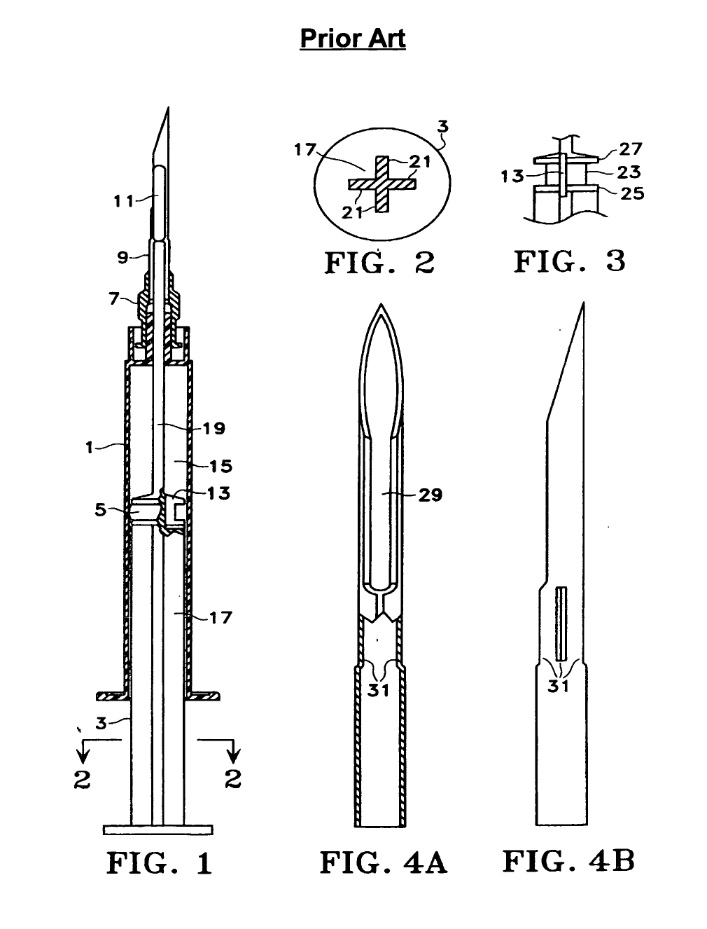

### DETAILED DESCRIPTION OF THE INVENTION

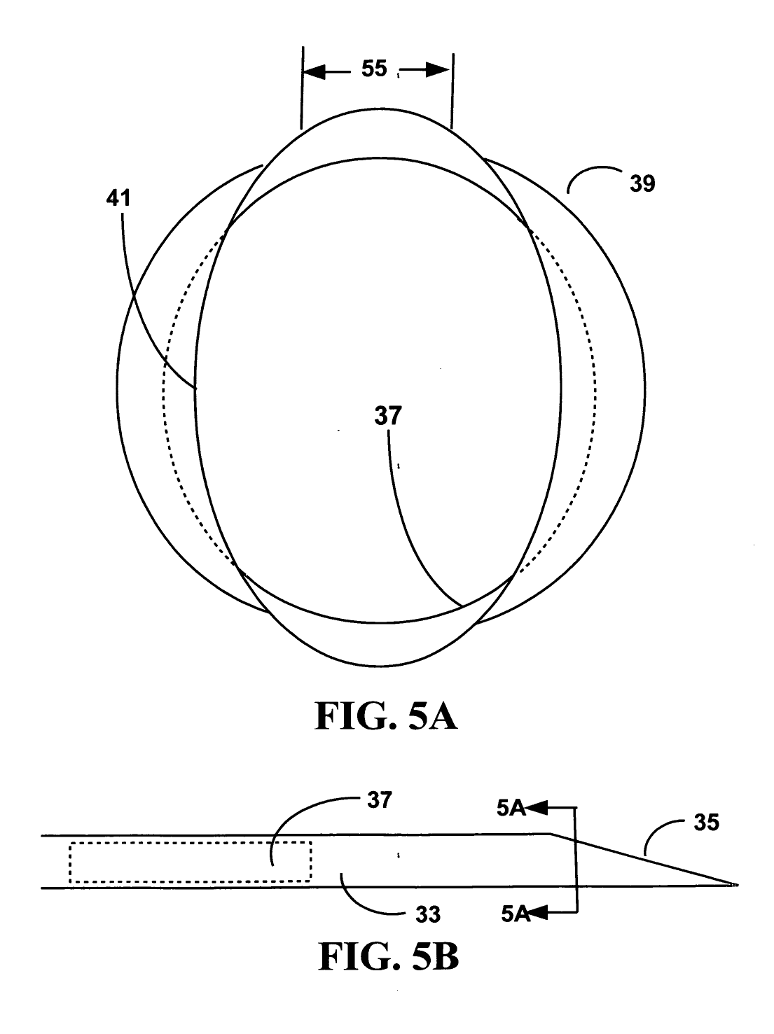

The invention is a cannula wherein the implant object is inserted into the cannula at the proximal end and is prevented from sliding out at the distal end by a structural impediment at the bevel site. The structural impediment can be achieved in a variety of ways. Changing the cross-sectional shape of the cannula from circular to oval at the bevel site is one possible way. A preferred embodiment of the cannula is shown in FIG. 5.

The side view of the distal portion of cannula 33 with bevel 35 at the distal end is shown in FIG. 5B. Cylindrical implant object 37 is shown at rest in the distal portion after having been inserted from the proximal end of the cannula.

A cross-sectional view of cannula 33 from a point at the upper part of bevel 35 and looking toward the proximal end is shown in FIG. 5A. Cannula 33 has a circular shape 39 from the proximal end to bevel 35 and there transitions to an elongated shape 41. This transition prevents the implant object 37, after being inserted into the circular proximal end of cannula 33, from going any further than the elongated distal end (see FIG. 5A and the relative dimensions of the circular proximal end of cannula 39, transplant object 37, and the elongated distal end of cannula 41).

The elongated distal end 41 of the cannula is an impediment to the movement of the implant object out of the cannula's distal end. Thus, implant object 37 cannot accidentally slide out of the distal end of cannula 33 as the cannula assumes a variety of orientations prior to the execution of the implantation process. With the initiation of the implantation process by pressing on the plunger the attached push-rod makes contact with and applies a force to the proximal end of implant object 37 which in turn applies forces to the sides of the cannula that have transitioned into an elongated shape. As a result of the bevel 35 there is a gap 55 in the wall of cannula 33. As the force exerted by the push-rod increases, implant object 37 forces the sides of the elongated portion of cannula 33 apart, gap 55 increases, and the structural impediment to the passage of implant object 37 out of the distal end of cannula 33 is circumvented.

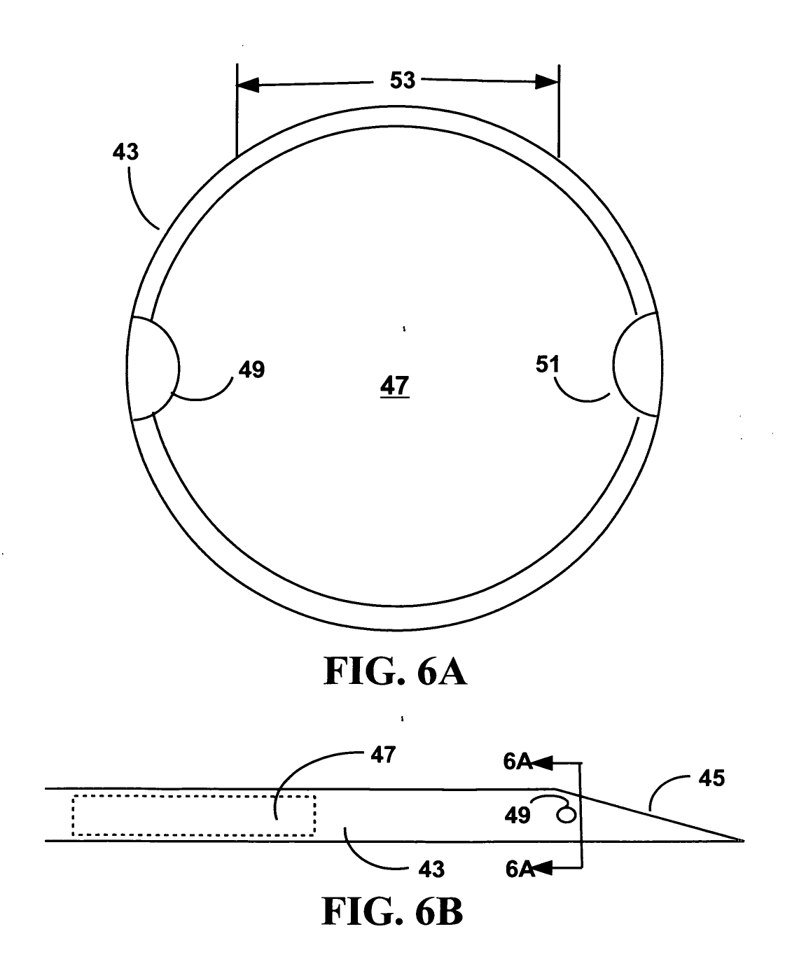

Another way of preventing an implant object from sliding out of the distal end of a cannula prior to the execution of the implantation process is shown in FIG. 6.

A side view of the distal portion of cannula 43 is shown in FIG. 6B The distal end of cannula 43 has a bevel 45. Implant object 47 is inserted into cannula 43 from the proximal end. The structural impediment which prevents the unintended passage of the implant object out of the cannula through the distal end are dimples on opposite sides of the cannula in the bevel region. Dimple 49 is shown in FIGS. 6A and 6B and dimple 51 is shown in FIG. 6A. The dimples are of sufficient size as to prevent the implant object 47 from sliding out through the distal end of the cannula, regardless of the orientation of the cannula.

When the implantation process is initiated by pressing on the plunger the attached push-rod makes contact with implant object 47 and applies a force to the proximal end of implant object 47 which in turn applies outward forces to dimples 49 and 51 and the cannula walls in which they are formed. The gap 53 in the cannula wall resulting from bevel 45 increases as the forces on the dimples increase. The two sides of the cannula spread apart, and ultimately the spread is sufficient for the implant object to pass by the dimples and out of the distal end of the cannula. Thus, the structural impediment blocking the exit of the implant object from the distal end of the cannula is circumvented by the execution of the implantation process.

What is claimed is:

1. A cannula adapted to receive an implant object into its interior at the proximal end, the cannula being attachable to an implantation device having a push-rod which enters the cannula at its proximal end, an operator utilizing the implantation device to make an incision in a body with the cutting edge of a bevel at the distal end of the cannula, the operator completing the implantation process by pushing on the push-bar thereby causing a force to be applied to the implant object resulting in the implant object moving from a position within the cannula out through the distal end and into the body, a structural impediment being incorporated in the bevel region of the cannula which prevents the implant object from falling out of the cannula at the distal end as a result of changes in cannula orientation prior to the execution of the implantation process, the structural impediment being automatically circumbented during the execution of the implantation process.

2. The cannula of claim 1 wherein the force exerted on the structural impediment by the implant object during the implantation process causes the structural impediment to move out of the way and allow the implant object to move to and out of the distal end of the cannula.

3. The cannula of claim 1 wherein the structural impediment is a change in cross-sectional shape of the cannula.

4. The cannula of claim 1 wherein the structural impediment is one or more dimples in the outer surface of the cannula.
